(19) United States
(12) Patent Application Publication
(10) Pub. No.: US 2005/0131372 A1
(43) Pub. Date: Jun. 16, 2005

(54) Absorbent articles with removable protective wing portions

(75) Inventors: Katherine C. Wheeler, Menasha, WI (US); Pat M. Exarhos, Appleton, WI (US); Christopher R. McKinney, Appleton, WI (US); Laura J. Walker, Appleton, WI (US); Manuel A. Torres, Draper, UT (US)

Correspondence Address: DORITY & MANNING, P.A., POST OFFICE BOX 1449, GREENVILLE, SC, 29602-1449, US

(73) Assignee: KIMBERLY-CLARK WORLDWIDE, INC.

(21) Appl. No.: 10/732,907

(22) Filed: Dec. 10, 2003

(51) Int. Cl. A61F 13/15; A61F 13/20

(52) U.S. Cl. 604/385.04

(57) ABSTRACT

An absorbent article, such as a feminine care product, includes a liquid permeable top cover, a generally liquid impermeable baffle, and an absorbent structure disposed between the top cover and outer cover. A wing structure is removably attachable to a garment facing side of the baffle and includes laterally extending flap portions having dimensions so as to wrap around the crotch portion of an undergarment. The garment facing side of baffle includes hook material defined in a pattern extending generally over the surface thereof, the hook material providing a primary attachment between the baffle and the undergarment. The wing structure has a body facing side of a hook compatible material and a garment facing side comprising hook material defined in a pattern extending generally over the surface thereof. The wing structure is variably positionable on and removably attachable over the entirety of the garment facing side of baffle.

### FIELD OF THE INVENTION

The present invention relates generally to the field of disposable absorbent articles, and more particularly to a feminine care article having laterally extending protective wings.

### BACKGROUND

Disposable absorbent articles intended to be worn in the crotch portion of an undergarment are well known. The basic form of such absorbent articles typically includes a bodyside liquid-permeable cover, a garment-side liquid-impermeable baffle, and an absorbent core positioned between the cover and the baffle. Such absorbent articles are now in wide use as sanitary napkins, panty shields, panty liners, and adult incontinence pads. While this invention is directed to all such products, for purposes of simplification, the invention will be described with reference to a sanitary napkin.

Present day commercial products have generally performed well, remaining in place and providing the user with ease of placement and removal. However, some of these products suffer from certain drawbacks. For example, the inner crotch surface of an undergarment, to which these products are typically adhered, is continually distorted, twisted and stretched due to the dynamics of the wearer. As a result, conventional adhesive attachments can detach causing the undesirable consequence of the sanitary napkin moving out of place. Further, while the sanitary napkin frequently reattaches, due to the continuing adhesive nature of the pressure sensitive adhesive, reattachment often places the sanitary napkin in an undesirable position wherein the sanitary napkin does not function properly. In an extreme case, the attachment of the adhesive also results in the adhesive folding over on itself and then becoming unavailable for reattachment.

In an effort to overcome the loss of protection due to the lack of close contact with the body of the wearer, and to ameliorate the above problem, disposable absorbent articles have been equipped with a pair of side panels, flaps or wings. The panels are intended to be folded around the edges of the wearer's undergarment. The panels potentially offer some functional improvements and advantages over a sanitary napkin without such panels. First, the panels protect the edges of the wearer's undergarment from being soiled by bodily fluids. Second, the panels help to stabilize the sanitary napkin from shifting out of place, especially when the panels are affixed to the underside of the panty. Each panel can be either integral with the cover and/or the baffle or can be fashioned from separate pieces of material and permanently adhered or bonded to the sanitary napkin at the longitudinal side edge or inward thereof.

Although the panels are beneficial for many users, they add significantly to the manufacturing process and costs of absorbent articles and have been problematic in their placement and retention in the crotch area of the undergarment. For example, conventional articles typically employ an adhesive on the garment facing side of the article body and panels which can be provided with a release liner to protect the adhesives from contaminants, such as dirt, and to keep the adhesive from sticking to the skin of the wearer and/or extraneous surfaces prior to use. When the wearer is ready to use the sanitary napkin, the release liners are peeled from the garment adhesives and the article is then pressed into the crotch region of the undergarment. The panels are then folded and secured on the underside of the panties, either affixed to the garment-facing side of the wearer's panty or affixed to themselves. Once the adhesives have been exposed and the article placed into the crotch region of the undergarment, it is generally not practical to remove and reposition the article. Unfortunately, it is highly probable that one or both of the panels may become inadvertently adhered to the undergarment body facing surface of the sanitary napkin or to each other in the process of affixing the article in the undergarment. Typically, in the process of extricating the adhered panel, the panel and/or the baffle may tear, ruining the sanitary napkin. Attention has thus been given to reducing the amount and locations of adhesives. For example, with KOTEX® sanitary napkins from Kimberly-Clark Corp., VELCRO® is used to attach the wings to each other.

It is also the case that permanently adhered or integrally formed panels are not optimally positioned on the article for all wearers and styles of undergarments. However, the panels cannot be repositioned without changing the location of the absorbent article itself, which may not be desired from performance, comfort, or external appearance aspects.

It may also be that wearers do not necessarily need or desire panels during certain periods of use, for example on lighter flow days, but would like to have the option for such panels if need be. Articles having permanently affixed or integrally formed panels do not serve this need.

WO 01/72254 A2 and WO 01/72254 A3 describe sanitary napkins that have a pair of integrally formed outwardly extending side flaps adapted to be folded around the edges of the crotch panel of a garment. The flaps overlap and attach to each other with a fastening device that permits subsequent release and reattachment to permit repositioning by the user. A longitudinal strip of adhesive is provided on the garment facing side of the article for attachment to a garment, and adhesive patches are provided on oppositely facing sides of the flaps for securing the flaps to each other after they have been folded around the crotch portion of the garment.

EP 1 245 209 A2 describes an absorbent article intended for use with thong-style undergarments that may include one or more removable wings for providing a more secure mechanism for attaching the article to the undergarment. The wing is a separate piece from the article and has a lower or “outer” garment-facing surface that may be coated with an adhesive or other separable fastener for attaching the wing to the garment after the wing is folded over. The outer surface of the article is similarly coated for attaching the wing to the article.

The present invention provides a unique absorbent article configuration that incorporates variably positionable and reusable panels or “wings” without the drawbacks mentioned above.

### SUMMARY

Objects and advantages of the invention will be set forth in part in the following description, or may be obvious from the description, or may be learned through practice of the invention.

Although the present invention has particular usefulness in the field of feminine care articles such as panty shields or liners and sanitary napkins, it should be appreciated that any manner of personal care absorbent article may benefit from the invention, including incontinence articles, and the like. All such uses are within the scope and spirit of the invention. For ease of description only, the working environment of the invention is assumed to be feminine care sanitary napkins.

In a particular embodiment of the invention, an absorbent article is provided for placement in the crotch region of an undergarment. The article includes conventional features such as a generally liquid permeable top cover, a generally liquid impermeable outer cover or baffle, and an absorbent structure disposed between the top cover and baffle. A readily removable and variably positional wing structure is provided for attachment to the garment facing side of the baffle. It should be understood that the term “wing structure” is meant to encompass a unitary structure having laterally extending flap portions, as well as individual wings or flaps that are separately positionable relative to the baffle. The flap portions have dimensions and a shape so as to wrap around the crotch portion of the undergarment.

The article utilizes unique placement of hook-type attachment material for the various purposes of attaching the baffle to the undergarment, attaching the removable wing structure to the baffle, and attaching the flap portions to the undergarment and/or to each other when folded around the crotch portion of the undergarment. In a particular embodiment, the garment facing side of the baffle includes hook material defined in an overall pattern generally over the entire surface area thereof, such hook material providing a primary attachment mechanism between the baffle and the undergarment. The pattern of hook material may be any desired continuous or discontinuous placement pattern, for example stripes, dashes, swirls, dots or island-like regions, etc. The amount and pattern of the hook material should be sufficient to ensure that the baffle remains secured in the crotch region of the undergarment.

The removable wing structure includes a body facing side of a hook compatible material, such as a nonwoven layer or material, that attaches to the baffle hook material when the wing structure is pressed against the baffle. Thus, the wing structure is readily attachable and removable from the baffle, and infinitely positionable laterally and longitudinally relative to the baffle as desired by the wearer due to the fact that the baffle hook material is provided in an overall general pattern and the hook compatible material is also provided generally over the surface area of the body facing side of the wing structure.

The wing structure also includes a garment facing side having hook material defined in a pattern generally over the entire surface area thereof, as described above with respect to the baffle. The pattern may be the same or different from that of the baffle. This hook material provides a mechanism for the flap portions of the wing structure to securely attach to the garment facing side of the undergarment and to each other once the article has been placed and the flaps folded around the crotch portion of the undergarment. Thus, once the wing structure has been attached to the article (baffle), the garment facing side of the article as defined by the baffle and the outer side of the wing structure presents an overall pattern of hook material generally over its entire surface area. The article will thus be retained in the undergarment far more securely than if a single area or region of adhesive were used, such as a longitudinal strip of adhesive along the center of the baffle, or individual regions of adhesive or hook material on the flap portions. Yet, even with such improved attachment, the wing structure is easily removable and variably positionable as required by the wearer.

The invention is not limited to any particular size or shape of the wing structure, particularly the flap portions. In one embodiment, the flap portions may have a generally trapezoidal shape with a longitudinal dimension of at least about one-half of that of the baffle.

As mentioned, the patterns of hook material on the baffle and wing structure are not limiting. It is desired that the patterns provide sufficient hook material to ensure secure attachment of the components without unnecessarily limiting breathability of the baffle. In a particular embodiment, the pattern may be generally stripes of the hook material having a width of about one-half of a distance between adjacent stripes. The stripes may be, for example, about one-eight inch and spaced apart about one-fourth inch. The regions of hook material in the patterns may be about 5% to about 75% of a total surface area of the baffle and the wing structure garment facing sides. In a particular embodiment, the hook material may be about 25% of the total surface area.

The hook material may be deposited on the baffle and wing structure materials by conventional methods, including by applying strips of hook material tape with adhesive, etc. In a particularly desirable embodiment, the hook material is extruded and laminated directly onto a base material used for the baffle or wing structure, as described in greater detail below.

Aspects of the invention will described below in greater detail by reference to particular embodiments, examples of which are illustrated in the figures.

### BRIEF DESCRIPTION OF THE FIGURES

FIG. 1 is a perspective component view of an absorbent article according to the invention.

FIG. 2 is an assembled component view of the absorbent according to FIG. 1

FIG. 3A is a partial cut-away body-facing view of the assembled article of FIG. 2.

FIG. 3B is a perspective in-use view of the article of FIG. 3A worn in the crotch region of an undergarment.

FIG. 4 is a cross-sectional view of the absorbent article taken along the lines indicated in FIG. 1.

FIG. 5 is a cross-sectional view of the absorbent article taken along the lines indicated in FIG. 2.

FIG. 6 is a garment-facing plan view of an alternate embodiment of an absorbent article according to the invention.

FIG. 7 is a top plan view of an alternate embodiment of the invention.

FIG. 8 is a top plan view of an alternate embodiment of the invention.

### DETAILED DESCRIPTION

The invention will now be described in detail with reference to particular embodiments thereof. The embodiments are provided by way of explanation of the invention, and are not meant as a limitation of the invention. For example, features described or illustrated as part of one embodiment may be used with another embodiment to yield still a further embodiment. It is intended that the present invention include these and other modifications and variations as come within the scope and spirit of the invention.

Referring to the Figures, in which like numerals indicate like parts throughout the several views, embodiments of a disposable absorbent article 10 are depicted. For purposes of illustration only, the disposable absorbent article 10 is exemplified as a sanitary napkin. One skilled in the art will readily understand the adaptability of the invention to other personal care and health care articles, such as, for example, panty liners, adult incontinence garments and the like that use a securement panel to position the article relative to a wearer's undergarment. Typically, a sanitary napkin is worn by a female to absorb body fluids, such as menses, blood, urine and other body excrements discharged during a menstrual period. The term “disposable”, as used herein, means that the absorbent article is discarded after a single use and is not intended to be laundered for subsequent reuse. Although the invention has particular usefulness for feminine care articles and, it should be appreciated that the invention is in no way limited to sanitary napkins in particular, or to feminine care articles in general.

Referring to the figures in general, an absorbent article 10 according to the invention includes a primary pad component 15. The pad has a generally liquid permeable top cover 14, a generally liquid impermeable outer cover or baffle 16, and an absorbent structure 22 disposed between the top cover 14 and baffle 16. The top cover 14 and baffle 16 are sealed together at their peripheral edges utilizing known techniques, such as, for example, gluing, crimping, hot-sealing or the like, the sealed edges defining an overall sealed peripheral edge 23 (FIGS. 4 and 5) of the pad 15. The pad 15 may take on various shapes, but will generally have opposite lateral sides and longitudinal ends. In the illustrated embodiments, the pad 15 has a well-known hourglass shape. Various other geometries of absorbent articles, including feminine care articles, are well known to those skilled in the art, and all such embodiments are within the scope and spirit of the invention.

The absorbent article 10, particularly the pad 15, is desirably provided with sufficient capacity to absorb and retain the intended amount and type of bodily exudate(s). The absorbent capacity is provided by the fluid retentive absorbent structure 22. The absorbent structure 22 can be any structure or combination of components which are generally compressible, conformable, non-irritating to a wearer's skin, and capable of absorbing and retaining liquids and certain body wastes. For example, the structure 22 may include an absorbent web material of cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In a particular embodiment, the absorbent web material is a matrix of cellulosic fluff, and may also include superabsorbent hydrogel-forming particles. The cellulosic fluff may comprise a blend of wood pulp fluff. One preferred type of fluff is identified with the trade designation NB 416, available from Weyerhaeuser Corp., and is a bleached, highly absorbent wood pulp containing primarily soft wood fibers. The absorbent materials may be formed into a web structure by employing various conventional methods and techniques. For example, the absorbent web may be formed with a dry-forming technique, an air forming technique, a wet-forming technique, a foam-forming technique, or the like, as well as combinations thereof. Methods and apparatus for carrying out such techniques are well known in the art.

The absorbent structure 22 can contain superabsorbent materials which are effective in retaining body fluids. As a general rule, the superabsorbent material is present in the absorbent web in an amount of from about 0 to about 90 weight percent based on total weight of the web. Superabsorbents have the ability to absorb a large amount of fluid in relation to their own weight. Typical superabsorbents used in absorbent articles, such as sanitary napkins, can absorb anywhere from 5 to 60 times their weight in body fluids. Superabsorbent materials are well known in the art and can be selected from natural, synthetic, and modified natural polymers and materials.

The absorbent web material may also be a coform material. The term “coform material” generally refers to composite materials comprising a mixture or stabilized matrix of thermoplastic fibers and a second non-thermoplastic material. Some examples of such coform materials are disclosed in U.S. Pat. No. 4,100,324 to Anderson, et al.; U.S. Pat. No. 5,284,703 to Everhart. et al.; and U.S. Pat. No. 5,350,624 to Georger. et al.; which are incorporated herein in their entirety by reference thereto for all purposes.

The absorbent web material utilized in the absorbent structure 22 is also selected so that the individual absorbent structure possesses a particular individual total absorbency depending on the intended article of use. For example, for infant care products, the total absorbency can be within the range of about 200-900 grams of 0.9 wt % saline, and can typically be about 500 g of saline. For adult care products, the total absorbency can be within the range of about 400-2000 grams of saline, and can typically be about 1300 g of saline. For feminine care products, the total absorbency can be within the range of about 7-50 grams of menstrual fluid, and can typically be within the range of about 30-40 g of menstrual fluid.

The absorbent structure 22 may be a multi-component and may include, for example, an intake layer or transfer delay layer in combination with the underlying absorbent web. Such configurations are well known to those skilled in the art.

The fluid permeable top cover 14 has an outwardly facing surface that may contact the body of the wearer and receive bodily exudate(s). The top cover 14 desirably is made of a material which is flexible and non-irritating to the wearer. As used herein, the term “flexible” is intended to refer to materials which are compliant and readily conform to the bodily surface(s) with which such materials are in contact, or materials which respond by easily deforming in the presence of external forces.

The top cover 14 is provided for comfort and conformability and functions to direct bodily exudate(s) away from the body, through the top cover 14 and toward the absorbent structure 22. The top cover 14 should retain little or no liquid in its structure so that the cover provides a relatively comfortable and non-irritating surface next to the tissues within the vestibule of a female wearer. The top cover 14 can be constructed of any woven or nonwoven material which is easily penetrated by bodily fluids which contact the surface of the cover. Examples of suitable cover materials include rayon, bonded carded webs of polyester, polypropylene, polyethylene, nylon, or other heat-bondable fibers, polyolefins, such as copolymers of polypropylene and polyethylene, linear low-density polyethylene, and aliphatic esters such as polylactic acid. Finely perforated film webs and net material can also be used. The cover may be apertured to increase its fluid intake capacity. A specific example of a suitable cover material is a bonded carded web made of polypropylene and polyethylene such as that used as cover stock for KOTEX® pantiliners and obtainable from Sandler Corporation, Germany. Other examples of suitable materials are composite materials of polymer and nonwoven fabric materials. The composite materials are typically in the form of integral sheets generally formed by the extrusion of a polymer onto a web of spunbonded material. The fluid permeable cover 14 can also contain a plurality of apertures formed therein which are intended to increase the rate at which bodily fluid(s) can penetrate through the cover and into the absorbent structure 22.

The top cover 14 may also be embossed with any desired embossing pattern to define embossed channels. Embossing techniques are well known to those skilled in the art. An embossing pattern not only creates an aesthetically pleasing surface, the channels facilitate intake of menses fluid. Menses will tend to flow along the densified edges of the channels rather than pool on contact points of the top cover 14.

The top cover 14 can be maintained in secured relation with the absorbent structure 22 by bonding all or a portion of the adjacent surfaces to one another. A variety of bonding methods known to one of skill in the art can be utilized to achieve any such secured relationship. Examples of such methods include, but are not limited to, the application of adhesives in a variety of patterns between the two adjoining surfaces, entangling at least portions of the adjacent surface of the absorbent with portions of the adjacent surface of the cover, or fusing at least portions of the adjacent surface of the cover to portions of the adjacent surface of the absorbent.

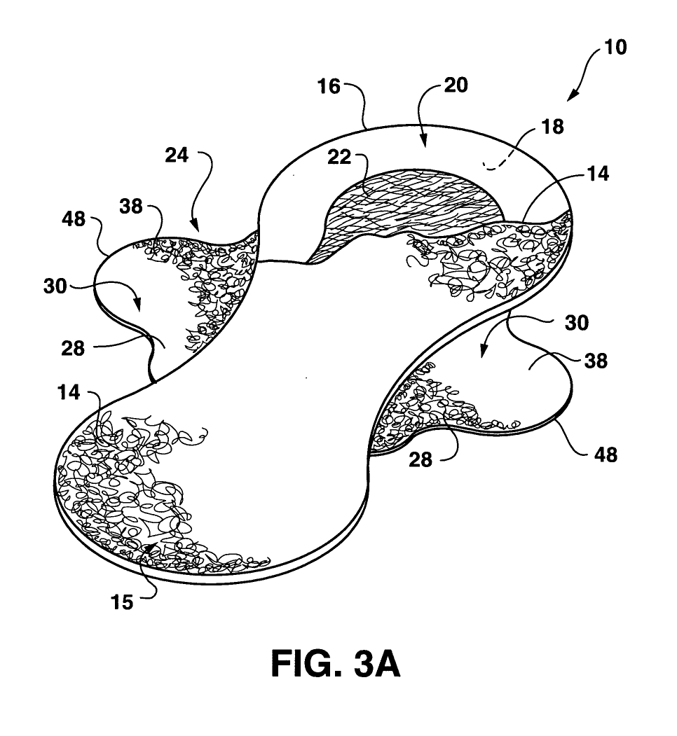

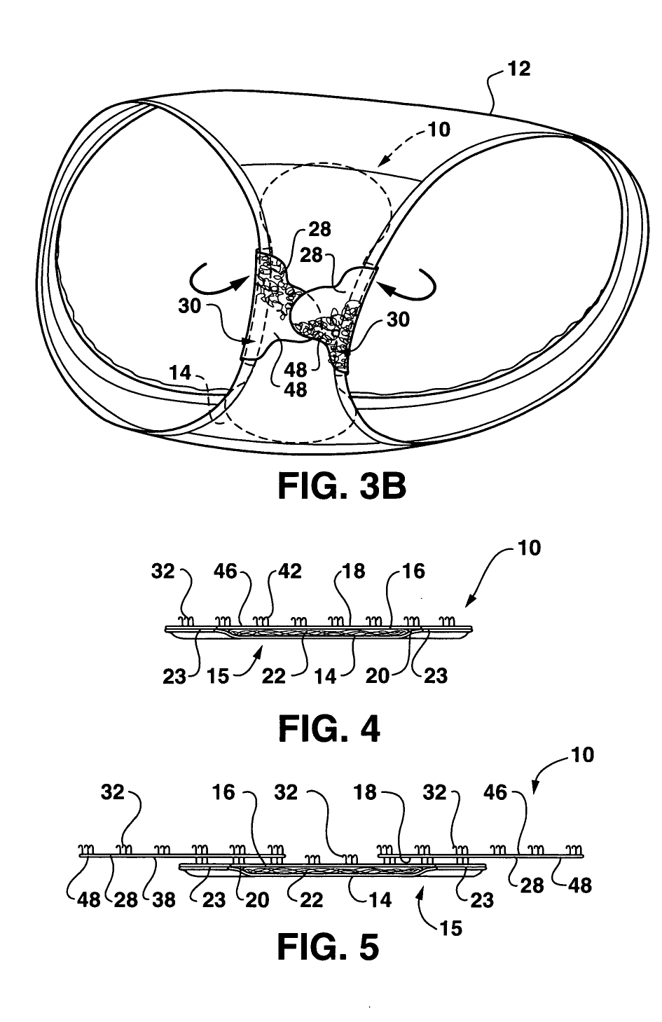

The baffle 16 has an outer garment facing side 18 and an inner body facing side 20 (FIG. 3), may be any one of a number of suitable liquid impermeable materials known in the art for use as outer covers or baffles in absorbent articles. Preferably, the baffle 16 will permit the passage of air and moisture vapor out of the pad 15 while blocking the passage of body fluids. A suitable material is a micro-embossed polymeric film, such as polyethylene or polypropylene, having a thickness of about 0.025 to 0.13 millimeters. Bicomponent films can also be used, as well as woven and nonwoven fabrics which have been treated to render them liquid impermeable. A specific example of a baffle material is a polyethylene film such as that used in KOTEX® pantiliners and obtainable from Pliant Corporation, Schaumburg, III., USA. The cover can be maintained in secured relation with the absorbent structure 22 by bonding all or a portion of the adjacent surfaces to one another. A variety of bonding methods known to one of skill in the art can be utilized to achieve any such secured relation. Examples of such methods include, but are not limited to, ultrasonic bonding, thermal bonding, or the application of adhesive materials in a variety of patterns between the two adjoining surfaces.

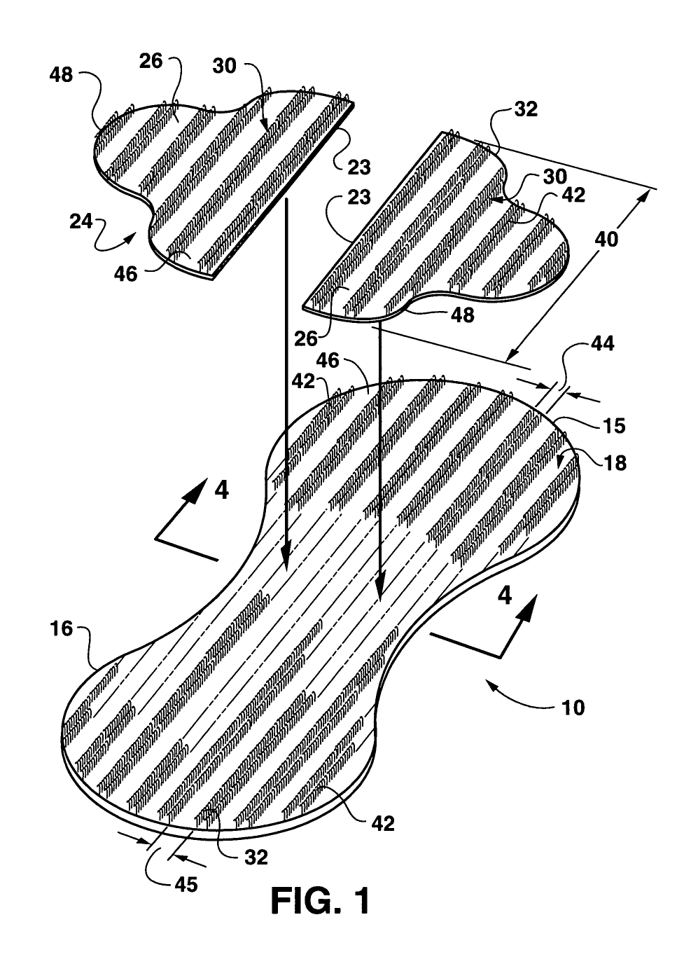

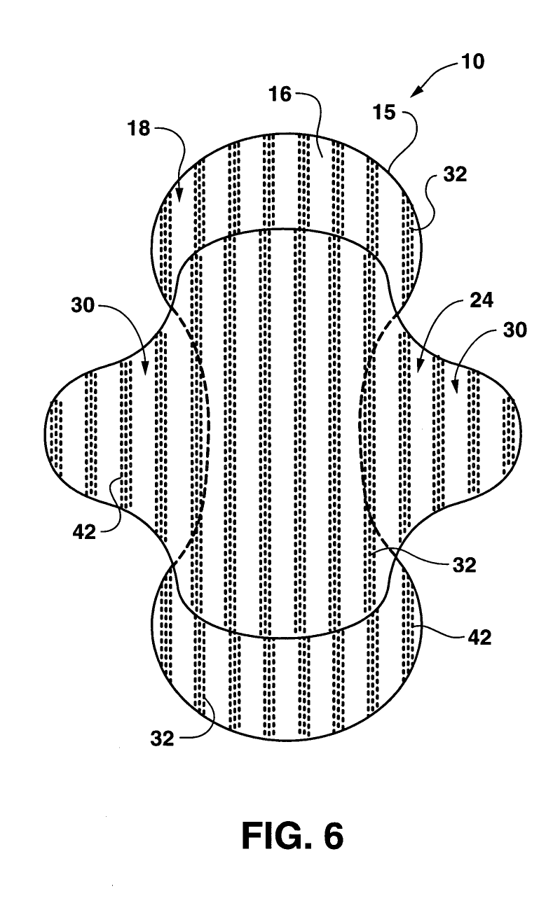

Referring to the figures in general, the article 10 includes a wing structure, generally 24, that is easily attachable and removable from the garment facing side 18 of the baffle 16. It should be appreciated that the term “wing structure” is used to denote any manner of structure the attaches to the pad 15 to define flap portions 30 having suitable dimensions and shape so as to extend around the crotch region of a wearer's undergarment 12 to aid in securing the article 10, as depicted in FIG. 4. In one particular embodiment, the wing structure 24 may include a single unitary structure having opposite laterally extending flap portions 30, as depicted in FIG. 6. In an alternate embodiment not illustrated in the figures, the wing structure 24 may include a member having a single flap portion 30. In a particularly useful embodiment illustrated in FIGS. 1, 2, and 5, the wing structure 24 includes separate flaps or wings 48 that are individually attachable and positionable on the baffle 16. The wings 48 include an inner longitudinal edge 23 and a laterally outboard flap portion 30. The wings 48 may have any desired length 40, for example about ½ of the overall length of the pad 15. In the figures, the wings 48 are illustrated as generally triangular or trapezoidal. It should be appreciated, however, that this is for illustration purposes only and the wings 48 may take on any shape, size, or suitable configuration.

The components of the wing structure 24, for example the separate wings 48, are readily attachable, removable, and variably positionable relative to the pad 15 depending on the needs or desires of the wearer. For example, depending on the type of undergarment 12, the wearer's flow conditions, visibility through an outer garment, comfort considerations, etc., the wearer may or may not desire the use of the optional wing structure 24 on any given day. The configuration of the article 10 according to the invention accommodates any such condition.

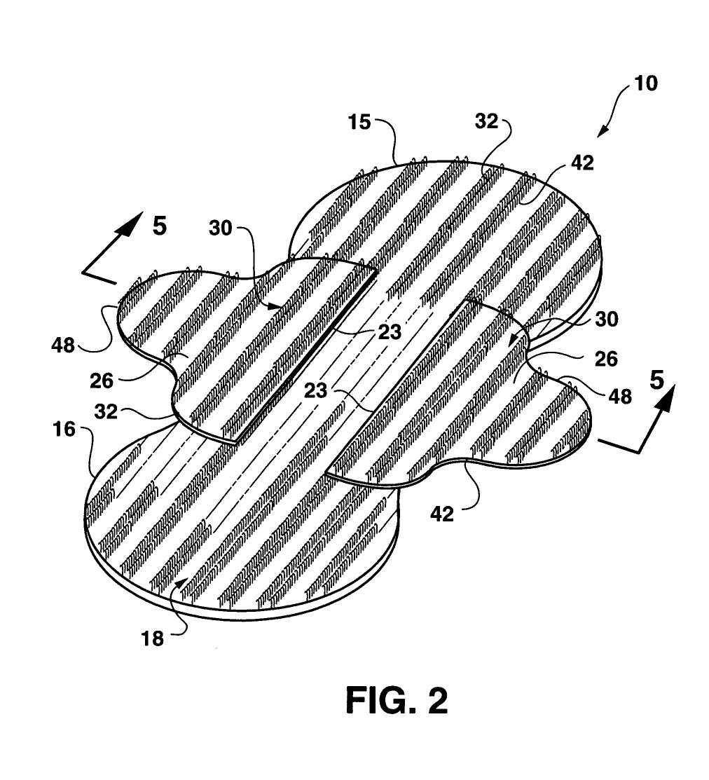

As described in greater detail below, the wing structure 24 includes a body facing side 28 that is variably positionable on the garment facing side 18 of the baffle 16 simply by pressing the respective sides together. In this manner, the wearer can position and reposition the structure 24 to obtain the best fit, protection level, and comfort based on their particular circumstances. To accomplish this, it is desirable that attachment of the wing structure 24 be possible over generally the entire surface area of the garment facing side 18 of the baffle 16. For example, referring to FIGS. 1 and 2, the wearer may desire to place one of the wings 48 at a forward longitudinal end of the pad 15, and the other wing 48 at the opposite end. The wings 48 can be positioned with their edges 23 relatively close together for undergarments having a relatively narrow crotch region, or further apart for wider crotch regions.

For attachment of the wing structure component or components 24 (i.e., the individual wings 48) to the baffle 16, a hook material 32 is provided in a pattern on the garment facing side 18 of the baffle 16. The hook material 32 is defined in a desired pattern over substantially the entire surface of the side 18. The particular design, shape, etc., of the pattern are not limiting features. The pattern should provide sufficient coverage such that the wing structure 24 may be positioned generally at any location relative to the body facing side 18 of the baffle 16. The amount of the hook material relative to the entire surface area of body facing side 18 may vary between about 5% to about 75%, with a desirably coverage being about 10% to about 50% of the surface area. The amount of coverage of the hook material 32 should not be so great that the baffle 16 is rendered vapor impermeable by the hook material.

As mentioned, the pattern of the hook material 32 may vary, but is desirably defined by deposits of hook material 32 interspaced with “bare” regions of the baffle material 16. In this way, the vapor permeability gradient of the baffle 16 is generally uniform over the surface area of the baffle 16. In a particular embodiment illustrated in FIGS. 1 through 6, the hook material is defined in a pattern of continuous longitudinal stripes 42 having a width 44 of, for example, about one-eight of an inch. The stripes 42 are interspaced with bands 46 of bare baffle material having a width 45 of, for example, one-quarter of an inch. Thus, the surface area of the hook material 32 is about one-half of that of the bare baffle material 16.

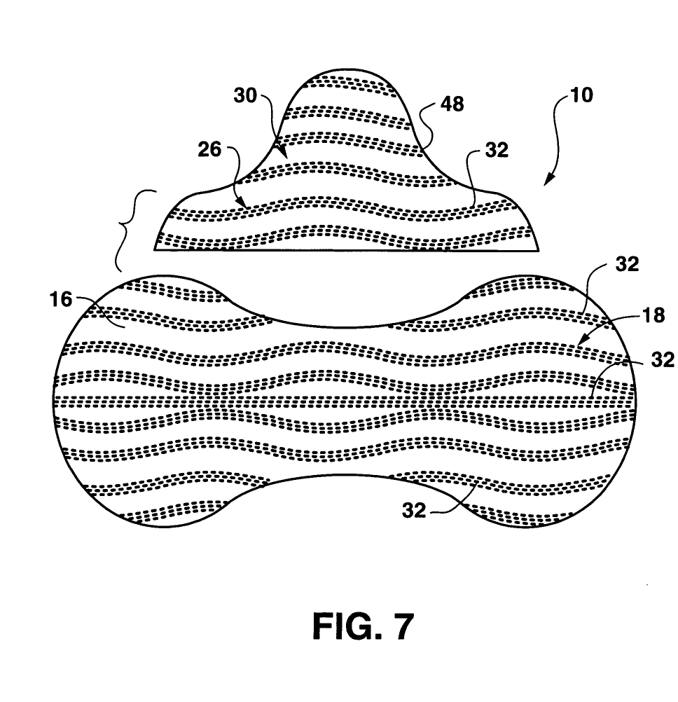

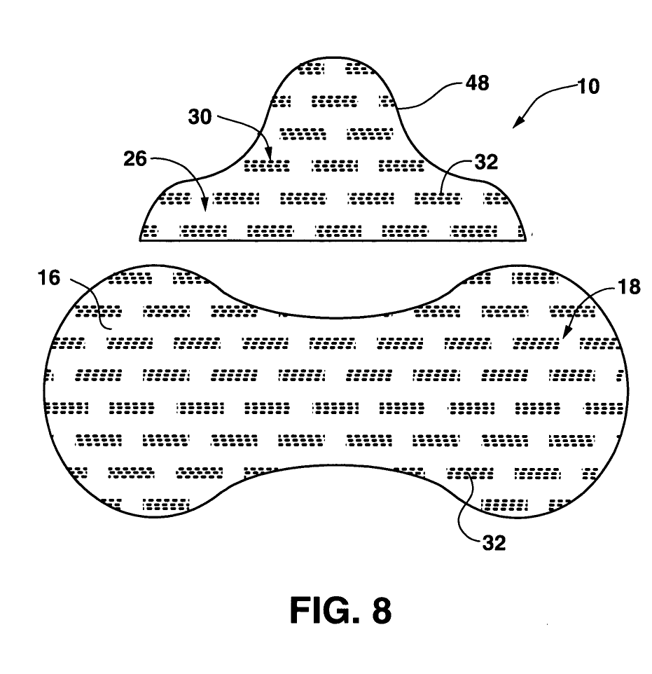

In an alternate embodiment, the pattern of hook material 32 may be defined in different complementary patterns. For example, in the embodiment of FIG. 7, the hook material 32 is defined as a series of serpentine stripes and center generally straight longitudinal stripes. In still an alternate embodiment, the hook material may be defined in a pattern of discontinuous deposits. For example, in the embodiment of FIG. 8, the hook material is defined by a pattern of broken stripes or alternating “dashes.” The discontinuous deposits may be, for example, island-like deposits such as circles, dots, etc., or deposits of irregular shapes. It should be appreciated that any desired pattern or shape of hook material deposits are within the scope and spirit of the invention.

The hook material 32 constitutes the male component of conventional hook-and-loop mechanical fastening systems that cooperates with a “loop” or loop-like material to define a releasable an re-attachable fastening system. Any number of commercially available and conventional micro-hook materials used in absorbent articles, including diaper attachment tabs, etc., may be used in the present invention. Conventional systems are, for example, available under the VELCRO® trademark. The hook element may be provided by a single-prong hook configuration, a multiple-prong hook configuration or by a generally continuous, expanded-head configuration, such as provided by a mushroom-head type of hook element. The many arrangements and variations of such fastener systems are collectively known in the art as hook-and-loop fasteners.

Examples of hook-and-loop fastening systems and components are described in U.S. Pat. No. 5,019,073 issued May 28, 1991 to T. Roessler et al., the entire disclosure of which is hereby incorporated by reference in a manner that is consistent herewith. Other examples of hook-and-loop fastening systems are described in U.S. patent application Ser. No. 366,080 entitled HIGH-PEEL TAB FASTENER, filed Dec. 28, 1994 by G. Zehner et al. which corresponds to U.S. Pat. No. 5,605,735; and U.S. patent application Ser. No. 421,640 entitled MULTI-ATTACHMENT FASTENING SYSTEM, filed Apr. 13, 1995 by P. VanGompel et al.; the entire disclosures of which are hereby incorporated by reference in a manner that is consistent herewith. Examples of fastening tabs constructed with a carrier layer are described in U.S. patent application Ser. No. 08/603,477 of A. Long et al., entitled MECHANICAL FASTENING SYSTEM WITH GRIP TAB and filed Mar. 6, 1996 which corresponds to U.S. Pat. No. 5,624,429 which issued Apr. 29, 1997, the entire disclosure of which is hereby incorporated by reference in a manner which is consistent herewith.

In a particularly desirable embodiment of the invention, the hook material 32 is directly extruded onto the baffle material 16 to define laminated regions of the hook material integral with the baffle material 16. Processes for directly extruding regions of a hook material directly onto a base material are described, for example, in U.S. Pat. Nos. 5,518,795; 5,260,015; and 5,744,080, all from Velcro Industries B.V. and incorporated herein in their entirety for all purposes. The extrusion process described in these patents may be used to directly deposit or embed the hook material with the baffle material 16 in any desired pattern. The baffle material 16 is selected so as to be suitable for the extrusion process and to support the embedded hook material 32. From a manufacturing perspective, rolls of the baffle material 16 having the hook material already embedded therein may be supplied directly into an in-line processing line for producing the pads 15. The extruded embedded regions of hook material 32 also reduce the thickness of the hooked regions as compared to conventional hook tape adhered to a base material.

Undergarments are typically made from various woven or non-woven materials that present an attachment surface for conventional hook materials. Thus, the hook material 32 on the garment facing side 18 of the baffle 16 may attach directly to the inner or body facing side of the wearer's undergarment, as illustrated in FIG. 3B, and provides the primary attachment mechanism between the pad 15 and the garment 12.

The wing structure 24 (i.e. unitary structure of FIG. 6 or separate wings 48 of the other Figs.) is formed of the same material as the baffle 16. However, liquid impermeability and breathability are generally not concerns with the wing structure 24 and, thus, a different less expensive material may also be used. Hook material 32 is provided on the garment facing side 26 of the wing structure 24 in a pattern that may be identical to the pattern of hook material 32 provided on the baffle 16, as shown in FIGS. 1 and 2, or may be defined in a different pattern. The hook material 32 on the wing structure 24 may be formed as described above with respect to the baffle 16. The pattern of hook material 32 is provided on the garment facing side 26 so as to releasably attach to the outer side of the undergarment 12 when the flap portions 30 are folded around the crotch region of the garment 12, as illustrated in FIG. 3B. Thus, the undergarment material provides the cooperating loop or loop-like surface for the wing structure hook material as well.

The body facing side 28 of the wing structure 24 is provided with a hook-compatible material 38 (FIG. 3A) for releasable attachment of the wing structure component or components 24 to the baffle 16 by merely pressing the components together. “Hook-compatible” material should be understood to encompass any material presenting a loop or loop-like surface for releasable attachment with the hook material 32. Suitable materials include, for example, a woven fabric, a nonwoven fabric, a knitted fabric, a perforated or apertured layer, and the like, as well as combinations thereof. Thus, the wing structure 24 may be formed primarily from any one or combinations of such materials.

Referring to FIGS. 2 and 6 for example, it can be seen that once the wing structure components are attached to the pad 15, an overall pattern of hook material 32 is presented on the garment facing side of the article 10 such that generally the entire surface area of the article 10 is attachable to the garment regardless of the presence of or position of the wing structure 24. This configurations greatly enhances attachment and versatility of the article 10.

With the present configuration, it should be understood that the flap portions 30 of the wing structure 24 need not overlap when folded around the crotch region of the undergarment 12, as depicted in FIG. 3B, because the flap portions 30 attach directly to the undergarment material. However, it may be desired in certain situations to position the wings 48 such that the respective flap portions 30 overlap around the crotch region for additional security. This may also be the case depending on the width of the garment's crotch region. In either case, such overlap is readily accommodated because the hook material 32 on the garment facing side 28 of the wing structure 24 readily attaches to the hook compatible material 38 provided on the body facing side of the flap portions 30 in the overlapped region.

It should be appreciated by those skilled in the art that various modifications and variations can be made to the embodiments of the absorbent article described herein without departing from the scope and spirit of the invention as set forth in the appended claims and equivalents thereof.

1. An absorbent article for placement in the crotch region of an undergarment, comprising:
a generally liquid permeable top cover;
a generally liquid impermeable baffle;
an absorbent structure disposed between said top cover and said baffle;
a wing structure removably attachable to a garment facing side of said absorbent article, said wing structure comprising laterally extending flap portions having dimensions so as to at least partially encircle the crotch portion of the undergarment;
said garment facing side of said absorbent article comprising hook material defined in a pattern extending generally over the surface thereof, said hook material providing a primary attachment between said absorbent article and the undergarment;
said wing structure having a body facing side of a hook compatible material and a garment facing side comprising hook material defined in a pattern extending generally over the surface thereof;
wherein said wing structure is variably positionable on and removably attachable to the entirety of said garment facing side of said absorbent article and, once said wing structure is attached, generally the entire surface area of said garment facing side of said absorbent article and said attached wing structure presents an overall pattern of hook material for attachment to the undergarment.

2. The absorbent article as in claim 1, wherein said wing structure comprises a unitary structure having laterally oppositely extending flap portions.

3. The absorbent article as in claim 1, wherein said wing structure comprises a pair of unattached flap portions, each said flap portion individually attachable to said absorbent article.

4. The absorbent article as in claim 2, wherein said flap portions have a longitudinal dimension of at least about one-half of that of said absorbent article.

5. The absorbent article as in claim 1, wherein said baffle and said wing structure of formed of a different material.

6. The absorbent article as in claim 1, wherein said pattern of hook material is defined on said baffle and matches said pattern of hook material on said wing structure.

7. The absorbent article as in claim 6, wherein said patterns of hook material comprise longitudinally disposed stripes of said hook material.

8. The absorbent article as in claim 7, wherein said stripes of said hook material have a width of about one-half of a distance between adjacent said stripes.

9. The absorbent article as in claim 6, wherein said hook material in said patterns comprises between about 5% to about 75% of a total surface area of said baffle and said wing structure garment facing sides.

10. The absorbent article as in claim 6, wherein said hook material in said patterns comprises about 25% of a total surface area of said baffle and said wing structure garment facing sides.

11. The absorbent article as in claim 6, wherein said patterns comprise discontinuous regions of said hook material.

12. The absorbent article as in claim 1, wherein said body facing side of said wing structure comprises a hook compatible nonwoven material.

13. The absorbent article as in claim 1, wherein said pattern of hook material is defined on said baffle, and said baffle and said wing structure comprise a base material with said regions of hook material deposited thereon.

14. The absorbent article as in claim 13, wherein said regions of hook material are laminated to said base material.

15. The absorbent article as in claim 14, wherein said hook material is formed and laminated to said base material in a single continuous processing step.

16. The absorbent article as in claim 1, wherein said pattern of hook material on said garment facing side of said absorbent article is different from said pattern of hook material on said wing structure.

17. The absorbent article as in claim 1, wherein said article is one of a sanitary napkin, panty liner, and adult incontinent article.

18. An absorbent article for placement in the crotch region of an undergarment, comprising:
a cover;
a generally liquid impermeable and vapor permeable baffle having a garment facing side;
an absorbent structure disposed between said top cover and said baffle;
a pair of wings removably attachable to said garment facing side of said baffle so as to at least partically encircle the crotch portion of the undergarment and attach to each other;
said garment facing side of said baffle and a garment facing side of said wings comprising a hook material provided thereon in a pattern such that said garment facing sides are attachable to the undergarment by way of said hook material;
said wing structure having a body facing side comprising a hook compatible material and;
wherein said wing structure is variably positionable on and removably attachable to said garment facing side of said baffle and, once said wing structure is attached, a pattern of said hook material is presented on said garment facing sides of said baffle and said attached wings.

19. The absorbent article as in claim 18, wherein said wings have generally the same shape.

20. The absorbent article as in claim 18, wherein said pattern of hook material on said baffle matches said pattern of hook material on said wings.

21. The absorbent article as in claim 20, wherein said patterns of hook material comprise longitudinally disposed and laterally spaced stripes of said hook material.

22. The absorbent article as in claim 21, wherein said stripes of said hook material have a width of about one-half of said lateral spacing between said stripes.

23. The absorbent article as in claim 18, wherein said hook material comprises between about 5% to about 75% of a total surface area of said baffle and said wings garment facing sides.

24. The absorbent article as in claim 18, wherein said patterns of hook material comprises discontinuous regions of said hook material.

25. The absorbent article as in claim 18, wherein said body facing side of said wings comprises a hook compatible nonwoven material.

26. The absorbent article as in claim 18, wherein said baffle and said wing structure comprise a base material with said hook material laminated to said base material.

27. The absorbent article as in claim 18, wherein said article is one of a sanitary napkin, panty liner, and adult incontinent article.
